(19) United States
(12) Patent Application Publication
(10) Pub. No.: US 2015/0018765 A1
(43) Pub. Date: Jan. 15, 2015

(54) INFUSION SET THAT PREVENTS AIR ENTRY INTO INFUSION TUBING

(71) Applicant: Welford Manufacturing (M) SDN BHD, Selangor (MY)

(72) Inventors: Thean Fook WONG, Shah Alam (MY); Wen Wvei WONG, Shah Alam (MY)

(21) Appl. No.: 14/325,567

(22) Filed: Jul. 8, 2014

(30) Foreign Application Priority Data
Jul. 9, 2013 (MY) PI2013002604

(51) Int. Cl. A61M 5/38; A61M 5/168; A61M 5/14

(52) U.S. Cl. CPC A61M 5/38; A61M 5/1411; A61M 5/16813
USPC 604/126

(57) ABSTRACT

The present invention relates to an infusion set (10) that provides for continuous air free delivery of liquid to patients undergoing infusion procedures. In particular, the invention provides for an infusion set (10) that prevents air entering the infusion tubing (40) through the drip chamber (20) by using a hydrophilic membrane (21) having a defined wetting time and a defined mean pore size that will provide a defined bubble pressure point under gravitational pull to permit continuous air free infusion liquid flow there-through. The invention further provides for a clip (50) that once closed, no air bubble is able to pass through the hydrophilic membrane (21) due to the adjustment of the roller clamp (60) along the infusion tubing (40) thereby ensuring safe replacement of the infusion bottle during multiple infusion procedures, thus preventing the occurrence of air embolism in patients especially when the drip chamber (20) is empty.

### TECHNICAL FIELD

The present invention relates to an infusion set that provides for continuous air free delivery of liquid to patients undergoing infusion procedures. In particular, the invention provides for an infusion set that prevents air entering the infusion tubing through the drip chamber by using a hydrophilic membrane having a defined wetting time and a defined mean pore size that will provide a defined bubble pressure point under gravitational pull to permit continuous air free infusion liquid flow there-through. The invention further provides for a clip that once closed, no air bubbles is able to pass through the hydrophilic membrane due to the adjustment of the roller clamp along the infusion tubing thereby ensuring safe replacement of the infusion bottle during multiple infusion procedures, thus preventing the occurrence of air embolism in patients especially when the drip chamber is empty.

### BACKGROUND

Intravenous infusion procedure is commonly used in hospital to deliver liquid (for example, saline solution, glucose solution, medicine, etc.) to patients seeking treatment. In hospitals, the intravenous infusion devices used are either gravity or pump driven. Gravity driven infusion devices typically include a conventional infusion line comprising a drip chamber with or without an air stop hydrophilic membrane at its lower end, a length of tubing designed to connect the lower end of the drip chamber to a connector, the connector is then connected via conventional access means such as a needle, a catheter or the like to a patient. The upper end of the drip chamber has a spike which is used to pierce a bottle containing a specified infusion liquid. The whole assembly comprising the bottle, the spike and the drip chamber is hung on a hanger to provide sufficient height so that the infusion liquid is pulled by gravitational force to allow the infusion liquid to flow downward towards the patient. A safety valve, for example a roller clamp, which can close the infusion tubing and also for regulating the rate of flow of the infusion liquid is provided on the infusion tubing between the drip chamber and the connector. The roller clamp is also used to close the infusion tubing as soon as the bottle containing the liquid is approaching empty or is empty.

Various problems are associated with infusion devices with or without air stop membrane. One of the problems commonly found in normal infusion devices without air stop membrane is that certain amount of air in the form of bubbles often gets into the blood circulation accidentally during the infusion procedure and which will lead to air embolism. Depending on the size of such air embolism, various complications suffered by the patient may include chest pain, wheezing, breathlessness, stroke, tachycardia (fast heart beat), sudden loss of consciousness leading to death, etc. Besides facing possible negligence claims brought against the hospitals by the patients, more commonly, hospitals will incur additional treatment cost in the case of complication due to air embolism.

Entry of air into the infusion tubing of an infusion set without air stop membrane can be caused mainly by the following ways:

(a) Improper priming of the infusion line at the start of the infusion procedure;

(b) During the infusion process itself, due to the fast dripping of the liquid;

(c) When the infusion bottle runs empty and the infusion process is not stopped; and

(d) When the infusion set is placed improperly or at a slant position during an emergency.

To counter this, medical staff normally will continuously monitor the infusion liquid level for presence of air bubbles in the infusion tube, and manually push the air bubbles out of the infusion tube if air bubbles are found. This practice of manually pushing out the air bubbles in the infusion tube takes up much of the medical staff time. In case where re-priming is needed, it also increases the chance of catheter infection. All these additional tasks undertaken by the medical staff will also increase the cost of providing healthcare to the patient.

To prevent air from entering the infusion tubing highlighted above, various methods have been used. One of the methods used is by incorporating an air stop membrane into the infusion set. However in a commercially available infusion set with air stop membrane, improper priming of the infusion line will still result in air bubbles being sucked into the infusion line due to the roller clamp adjustment during setup and during changing of a new infusion bottle. This is due to the design of the roller clamp which could cause air embolism when it is used alone in an infusion device with an air stop membrane. The roller clamp works on the principle that the infusion tube is gradually pressed by the roller clamp to the correct amount of compression to provide the required flow rate. Because the roller clamp presses and moves along the surface of the infusion tube in both directions during adjustment and locking, extra positive or negative pressures will be generated in the liquid which will ultimately act on the lower surface of the membrane. The amount of this extra pressure generated would depend on the speed that the roller clamp is moved. Occasionally it could create a suction pressure which is larger than the bubble pressure point of the wetted membrane and air bubbles would be sucked through the membrane into the infusion tube.

During the setting up of a new infusion device, the roller clamp will be first pushed into the locking position and the piercing spike will be pushed into the infusion bottle. The drip chamber will then be squeezed for a few times to force the liquid to flow down from the bottle into the drip chamber to the required level. The roller clamp is then released from its locking position to fully prime the line until all the air bubbles are completely flushed out. The roller clamp will then be locked again so that it could be connected to a needle or a catheter. After a firm connection to the needle or catheter has been established, the roller clamp will be opened and finally be adjusted to give the required drip rate.

At the end of the infusion process, the bottle will be empty and the liquid in the drip chamber will go down slowly and stop right above the upper surface of the membrane. The totally wetted membrane will act as a barrier to air so that no air bubble could pass through and enter into the infusion line so long as the roller clamp is not adjusted. At this moment, the liquid height in the infusion line from the membrane to the patient should remain unchanged. If multiple infusions are required, the same infusion device could be pulled out from the empty bottle and be re-inserted into a new bottle via the piercing spike without altering the roller clamp adjustment.

However in actual practices, due to the habitual procedures in setting up a new line, the medical staff may accidentally push the roller clamp into its locking position again before pulling out and re-inserting the piercing spike into a new bottle. This very pushing action on the roller clamp will generate a suction pressure on the lower surface of the wetted membrane of an empty drip chamber. If the suction pressure generated is higher than the bubble pressure point of the wetted membrane which is only slightly bigger than 30 kPa (0.3 bar), air bubbles will be sucked through the membrane into the infusion line without being noticed. Consequently air embolism could still be introduced into the patient's body once infusion is resumed with a new bottle of liquid.

Another problem that is common in a commercially available infusion set with an air stop membrane is that occasionally after setting up a new infusion device and filling up the drip chamber to the required level with the infusion liquid, the liquid in the drip chamber could not start to flow by gravitational pull even when the roller clamp is fully open. As indicated in the manufacturers' instructions for use of such infusion device, when this happens, the user or the medical staff is requested to create a suction action on the membrane by rolling the roller clamp in both directions on the infusion tube or manually pumping the drip chamber to start the infusion. These milking or pumping actions may cause inconvenience to the medical staff.

Another method of preventing air from entering the tubing is to have a pump driven intravenous infusion set with an air-liquid separator of the type comprising a containing body forming two adjacent chambers separated by a hydrophilic membrane and the containing body has an inlet aperture for a fluid comprising liquid and gas particles. The liquid can pass through the hydrophilic membrane and emerge through an outlet aperture. The gas which reaches the first chamber is discharged through a secondary aperture positioned upstream of the hydrophilic membrane, and at least one hydrophobic membrane is being used at these apertures to prevent the liquid from passing through.

The device which has been described above allows the fluid containing gas particles, to be separated into two parts, namely a liquid portion which emerges from the outlet aperture provided in the second chamber, and a gas portion which is released through the secondary aperture provided in the first chamber. It should be noted that the air separator device which has been described does not require a constant presence of liquid stagnating within it in order to separate the gas. In other words, the fluid passing through the separation device is continuously divided into the liquid, which continues along the line, and the gas, which is discharged to the exterior.

Although the use of air-liquid separator in a pump driven infusion set has significantly helped reduce air embolism in patients, the fluid collection chamber can only separate air from the liquid only when a minimum quantity of liquid is present in the chamber. If the liquid in the collection chamber is used up and this inevitably occurs after a certain time when the infusion liquid has been used up, unless the infusion pump is stopped at the correct time, there will be a transfer of air or gas towards the patient.

To overcome the problems described above, the inventors of the present invention have found that air bubbles can be prevented from entering the infusion tubing through the drip chamber of an infusion set by using a hydrophilic membrane having a defined wetting time and a defined mean pore size that will provide a defined bubble pressure point under gravitational pull to permit continuous air free infusion liquid flow there-through. The invention further provides for a clip that once closed, will prevent air bubbles from being sucked through the hydrophilic membrane due to the adjustment of the roller clamp along the infusion tubing thereby ensuring safe replacement of the infusion bottle during multiple infusion procedures, thus preventing the occurrence of air embolism in patients especially when the drip chamber is empty.

These and other aspects of the present invention will be better understood in the detailed description below.

### SUMMARY OF THE INVENTION

According to the first aspect of the present invention, there is provided an infusion set (10) comprising a drip chamber (20), said drip chamber has a spike (30) on the upper end, an infusion liquid outlet (22) at the lower end of the drip chamber, said infusion liquid outlet (22) is covered with a hydrophilic membrane (21), said hydrophilic membrane (21) effective when wet; an infusion tubing (40) connecting the lower end of the drip chamber (20) via an infusion liquid outlet (22) to a standard connector (70); a roller clamp (60) located between the drip chamber (20) and the standard connector (70), said standard connector is connected to a patient via conventional connecting means, characterized in that the hydrophilic membrane (21) has a defined wetting time and a defined mean pore size that will provide a defined bubble pressure point under gravitational pull to permit continuous air free infusion liquid flow there-through. In a preferred embodiment of the present invention, the defined wetting time is less than or equal to 10 seconds when the height of the water above the dry membrane is between 0.5 cm to 4 cm, the defined mean pore size of the hydrophilic membrane (21) is greater than or equal to 5 microns, and the defined bubble pressure point is greater than or equal to 30 kPa (0.3 bar) measured with water.

According to a second aspect of the present invention, there is provided an infusion set (10) characterized in that there is provided a clip (50) located between the drip chamber (20) and the roller clamp (60). When the clip (50) is closed, no air bubbles will pass through the wetted membrane (21) when the roller clamp (60) is adjusted along the infusion line. This will ensure safe replacement of the infusion bottle without inducing air bubbles into the infusion line during multiple infusion procedures when the drip chamber (20) is empty.

According to a third aspect of the present invention, there is provided an infusion set (10) characterized in that the flow rate of water is between 50 ml per minute to 300 ml per minute under gravitational pull when the clip (50) and the roller clamp (60) is fully opened.

According to a fourth aspect of the present invention, there is provided an infusion set (10) characterized in that the hydrophilic membrane (21) is made of polysulfone or polyethersulfone.

Further advantages of the present invention will now be described with reference to the accompanying drawings below, but not exclusive, embodiment of an infusion set using a hydrophilic membrane with a defined wetting time of less than or equal to 10 seconds when the height of the water above the dry membrane is between 0.5 cm to 4 cm, a defined mean pore size of between 1 micron to 10 microns, a defined bubble pressure point of between 10 kPa (0.1 bar) to 50 kPa (0.5 bar) measured with water and giving a desired flow rate of water of 50 ml per minute to 300 ml per minute under gravitational pull, thereby permitting continuous air free infusion liquid flow to the patients in need of such liquid.

### DESCRIPTION OF THE DRAWINGS

FIG. 1 is a schematic drawing of the infusion set of the present invention.

FIG. 2 is an exploded schematic drawing of the drip chamber of the infusion set of the present invention.

### DETAILED DESCRIPTION OF THE INVENTION

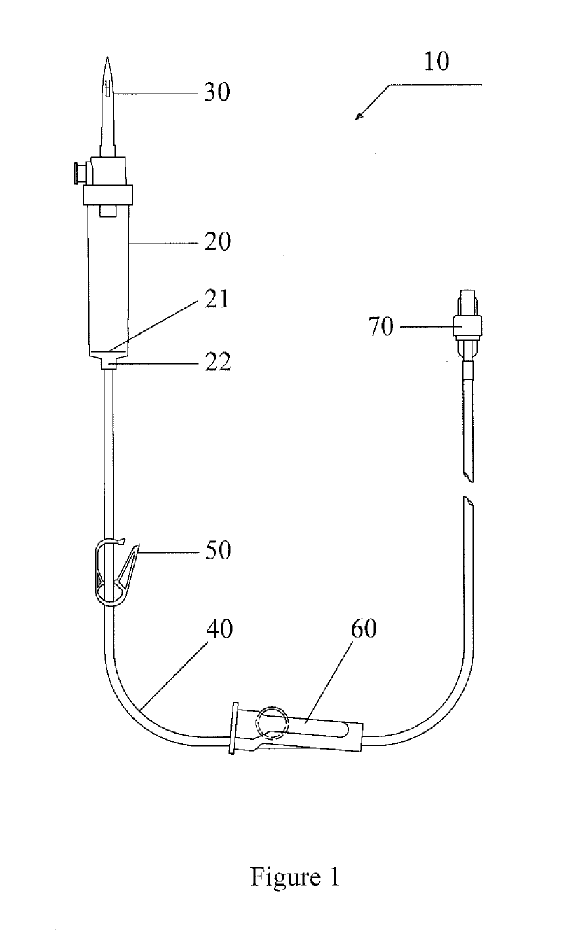

As shown in FIG. 1, the infusion set (10) comprises a drip chamber (20), said drip chamber (20) has a spike (30) on the upper end and a hydrophilic membrane (21) at its lower end, an infusion tubing (40) of sufficient length connecting the lower end of the drip chamber (20) to a standard connector (70), a roller clamp (60) is located between the drip chamber (20) and the standard connector (70), and a clip (50) is located between the drip chamber (20) and the roller clamp (60) along the length of the infusion tubing (40).

Referring to FIG. 1, an infusion line (40) is connected to the drip chamber (20). At the patient end of the infusion line, a standard connector (70) is provided so that a needle or a catheter could be connected to the patient. A flow adjustment device in the form of a roller clamp (60) is provided in the infusion line. The roller clamp (60) could be gradually adjusted so that the infusion tube could be squeezed proportionally to change the infusion rate.

Referring to FIG. 1, a clip (50) is also provided just beneath the bottom of the drip chamber (20) or in the region between the drip chamber (20) and the roller clamp (60). The clip (50) stops the flow of infusion liquid instantly by a clipping action. Once the clip (50) is closed, the roller clamp (60) can be adjusted along the infusion line without fear that there will be any inadvertent rise of the pressure in the infusion tubing due to the rolling action of the roller clamp (60). As a result, air bubbles are prevented from being sucked through the wetted hydrophilic membrane (21) of an empty drip chamber (20) by any movement of the roller clamp (60) or when the infusion bottle is changed for multiple infusion procedures.

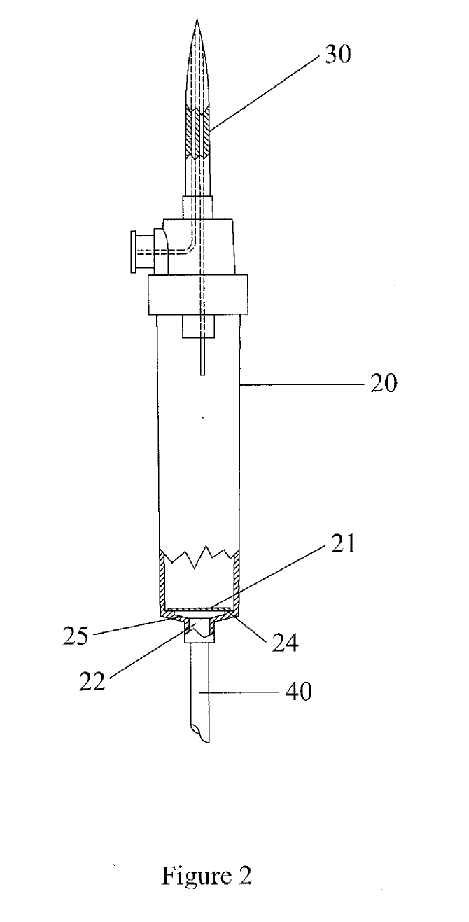

Referring to FIG. 2, the drip chamber (20) is equipped with a piercing spike (30) on the upper end. The piercing spike will be pushed into the seal of an infusion bottle during infusion therapy so that the infusion solution will be pulled by gravity into the drip chamber. The lower end of the drip chamber is designed with a flat sealing ring (24) and a funnel shaped connector (25). A hydrophilic membrane (21) is sealed onto the sealing ring (24) and covering the infusion liquid outlet (22) so that air bubbles and particles could be prevented from entering the body of the patient during infusion therapy.

In the present invention, the mean pore size of the hydrophilic membrane (21) used is between 1 micron to 10 microns, preferably 5 microns. Due to the hydrophilicity and the mean pore size of the hydrophilic membrane (21), the bubble pressure point of the hydrophilic membrane (21) is greater than 10 kPa (0.1 bar) measured with water. Preferably the bubble pressure point is greater than or equal to 30 kPa (0.3 bar) measured with water.

The bubble pressure point of the hydrophilic membrane is defined as the minimum pressure required to push the trapped wetting liquid out of the holes and to allow first air bubble to appear. Generally, the smaller the holes in the hydrophilic membrane (i.e. smaller the pore size), the higher is the bubble pressure point. However, a small pore size would also mean a slower flow rate. Hence considerations must be made to have the correct mean pore size for the required bubble pressure point in order to provide an optimum flow rate for the current application. Based on our studies, the bubble pressure point of the required hydrophilic membrane should be at least 10 kPa (0.1 bar) and preferably higher than 30 kPa (0.3 bar) in our application measured with water.

Theoretically the hydrophilic membrane (21) could be made to provide a very high bubble pressure point so that the suction pressure generated by the roller clamp (60) could never suck any air bubbles through the infusion tubing to cause air embolism. But in physics, by increasing the bubble pressure point, the flow rate of the membrane would inevitably decrease markedly at the same time. Hence it is not practical to solve this problem by just increasing the bubble pressure point of the hydrophilic membrane alone.

In the present invention, the flow rate of the infusion set is dependent on the surface area of the hydrophilic membrane, the inner diameter of the infusion tubing and the pressure created by gravity pull. Accordingly, the bottom of the drip chamber (20) of the present invention has been designed with a small flat sealing ring (24) and a funnel shaped connector (25) to provide for a maximum flow area. When the said hydrophilic membrane (21) is sealed onto the sealing zone of the drip chamber (20), the flow rate achieved with the clip (50) and roller clamp (60) fully opened is at least 50 ml per minute measured with water under gravitational pull. Preferably the flow rate is in the range of 50 ml per minute to 300 ml per minute under the same conditions, and more preferably, the flow rate is between 150-250 ml per minute under the same conditions and even more preferably the flow rate is greater than or equal to 200 ml per minute under the same conditions.

It will be appreciated that different flow rates are required in different stages and situations in an infusion therapy such as the priming stage, the infusion stage and in an emergency case. Hence membranes with a higher flow rate will give more flexibility to the medical staffs. In the present invention, the inventors have found that by combining membranes having a mean pore size of 5 microns or larger pores and the surface area of drip chamber, the flow rate of water achievable is minimum 50 ml per minute under gravity pull when the clip (50) and the roller clamp (60) is fully opened. With this minimum flow rate of 50 ml per minute, the requirement that a flow rate in the range of 5 ml per hour to 250 ml per hour used in the infusion therapy in the hospital is met.

In the present invention, the hydrophilic membrane (21) used is made of polysulfone or polyethersulfone as the preferred materials. Our studies show that the said hydrophilic membrane (21) of the present invention when made using these materials has a higher hydrophilicity and hence the hydrophilic membrane will have a very low wetting out time under gravity. The wetting time is defined as the time taken for a dry membrane to become totally wetted so that the wetting liquid could start to flow through the hydrophilic membrane under normal gravitational pull.

The ability of a membrane to filter out air bubbles is due to its inherent hydrophilic property. When a membrane is hydrophilic, it has a tendency to attract water (a strong affinity for water) while restricting air from attaching to its surface. As a result, a fully wetted hydrophilic membrane will allow water to flow through while acting like a barrier to air bubbles passing across it. Under normal conditions, a more hydrophilic membrane would become totally wetted more easily and flow of water is also expected to start under gravity pull more readily. Based on our studies, when the drip chamber covered with the said dry hydrophilic membrane is filled with water to a height of between 0.5 cm to 4 cm above the membrane, the wetting time is less than or equal to 10 seconds i.e. water will start to flow under gravity pull in less than or equal to 10 seconds. On the other hand, when the drip chamber covered with the said dry hydrophilic membrane is filled with water to a height of greater than 4 cm above the membrane, the wetting time is less than or equal to 5 seconds, i.e. water will start to flow under gravity pull in less than or equal to 5 seconds. Hence with the present membrane setup, the fluid in the drip chamber will be ready to flow once it is filled up. No milking of the roller clamp is required to initiate the flow.

In the present invention, the said hydrophilic membrane (21) will automatically stop the fluid flow once the fluid in the drip chamber (20) is empty and as long as the membrane is still totally wet. As such no air will get into the infusion line during and at the end of the infusion procedure. Furthermore because the same device can be used for multiple infusions, the material cost and clinical waste is greatly reduced and this is one of the advantages of the present invention.

In cases where patients need to be transferred for emergency treatments, the infusion device together with the infusion bottle could be placed in awkward positions under critical situations. Under these situations, no air bubbles will get into the infusion line when the infusion set is placed in any orientation because the hydrophilic membrane (21) will automatically stop the fluid flow once the fluid in the drip chamber (20) is empty and as long as the membrane is still totally wet. Hence the said device would not pose any risk to patient during transportation and this is one of the advantages of the present invention.

To ensure that there is no inadvertent rise of the pressure in the infusion tubing due to adjustment of the roller clamp (60) that may pull the air bubbles through the hydrophilic membrane (21), a clip (50) is placed after the drip chamber (20) and before the roller clamp (60). The clip (50) will stop the flow of infusion liquid instantly when the clip is closed by clipping action. As a result, air bubbles are prevented from being sucked through the wetted hydrophilic membrane (21) of an empty drip chamber (20) by any movement of the roller clamp (60) or when the infusion bottle is changed for multiple infusion procedures. This represents another advantage of the present invention.

It will be appreciated that, although specific embodiments of the invention have been described herein for the purpose of illustration, various modifications may be made without deviating from the spirit and scope of the invention as defined in the following claims.

1. An infusion set (10) comprising:
a drip chamber (20), said drip chamber has a spike (30) on the upper end, an infusion liquid outlet (22) at the lower end of the drip chamber, said infusion liquid outlet (22) is covered with a hydrophilic membrane (21), said hydrophilic membrane (21) is effective when wet;
an infusion tubing (40) connecting the lower end of the drip chamber (20) via an infusion liquid outlet (22) to a standard connector (70);
a roller clamp (60) located between the drip chamber (20) and the standard connector (70), said standard connector is connected to a patient via conventional connecting means;
characterised in that the hydrophilic membrane (21) has a defined wetting time and a defined mean pore size that will provide a defined bubble pressure point under gravitational pull to permit continuous air free infusion liquid flow there-through.

2. An infusion set (10) as claimed in claim 1 characterized in that the hydrophilic membrane (21) has a wetting time of less than or equal to 10 seconds when the height of the water above the dry hydrophilic membrane (21) is between 0.5 cm to 4 cm.

3. An infusion set (10) as claimed in claim 1 characterized in that the hydrophilic membrane (21) has a wetting time of less than or equal to 5 seconds when the height of the water above the dry hydrophilic membrane (21) is greater than 4 cm.

4. An infusion set (10) as claimed in claim 1 characterized in that the defined mean pore size of the hydrophilic membrane (21) is between 1 micron to 10 microns.

5. An infusion set (10) as claimed in claim 4 characterized in that the defined mean pore size of the hydrophilic membrane (21) is greater than or equal to 5 microns.

6. An infusion set (10) as claimed in claim 1 characterized in that the defined bubble pressure point is between 10 kPa (0.1 bar) to 50 kPa (0.5 bar) measured with water.

7. An infusion set (10) as claimed in claim 6 characterized in that the defined bubble pressure point is greater than or equal to 30 kPa (0.3 bar) measured with water.

8. An infusion set (10) as claimed in claim 1 characterized in that there is provided a clip (50) located between the drip chamber (20) and the roller clamp (60).

9. An infusion set (10) as claimed in claim 8 characterized in that when the clip (50) is closed, no air bubble is able to pass through the hydrophilic membrane (21) when the roller clamp (60) is adjusted.

10. An infusion set (10) as claimed in claim 9 characterized in that when the clip (50) is closed, no air bubbles will be induced into the infusion tubing (40) during multiple infusion procedures.

11. An infusion set (10) as claimed in claim 1 characterized in that when the clip (50) and the roller clamp (60) is fully opened, the flow rate of water is at least 50 ml per minute under gravitational pull.

12. An infusion set (10) as claimed in claim 11 characterized in that the flow rate of water is between 50 ml per minute to 300 ml per minute under gravitational pull.

13. An infusion set (10) as claimed in claim 12 characterized in that the flow rate of water is greater than or equal to 200 ml per minute under gravitational pull.

14. An infusion set (10) as claimed in claim 1 characterized in that the hydrophilic membrane (21) is made of polysulfone.

15. An infusion set (10) as claimed in claim 1 characterized in that the hydrophilic membrane (21) is made of polyethersulfone.
